(19) United States
(12) Patent Application Publication
(10) Pub. No.: US 2017/0156409 A1
(43) Pub. Date: Jun. 8, 2017

(54) WRAPPING STRUCTURE

(71) Applicants: BOE TECHNOLOGY GROUP CO., LTD., Beijing (CN); BEIJING BOE OPTOELECTRONICS TECHNOLOGY CO., LTD., Beijing (CN)

(72) Inventors: Xiaoliang DING, Beijing (CN); Haisheng WANG, Beijing (CN); Xiaochuan CHEN, Beijing (CN); Weijie ZHAO, Beijing (CN); Shengji YANG, Beijing (CN); Rui XU, Beijing (CN); Lei WANG, Beijing (CN); Wei LIU, Beijing (CN); Changfeng LI, Beijing (CN); Hongjuan LIU, Beijing (CN); Yingming LIU, Beijing (CN); Tao REN, Beijing (CN)

(21) Appl. No.: 15/117,350

(22) PCT Filed: Nov. 5, 2015
(86) PCT No.: PCT/CN2015/093827

§ 371 Date: Aug. 8, 2016

(30) Foreign Application Priority Data
Jun. 29, 2015 (CN) 201510369810.9

(51) Int. Cl. A41B 13/06; A61B 5/00; A61B 5/11

(52) U.S. Cl. CPC A41B 13/06; A61B 5/11; A61B 5/746; A61B 5/683

(57) ABSTRACT

A wrapping structure is provided by the present disclosure, including a wrapping material configured to wrap an active object. The wrapping material includes a detection layer configured to monitor a motion of the active object wrapped by the wrapping material and convert the motion of the active object into an electric signal. An amplitude value of the electric signal is in direct proportion to a range and a frequency of the motion of the active object. The detection layer determines that the active object feels uncomfortable and sends a detection processing instruction, in the case that the amplitude value of the electric signal is not corresponding to a predetermined threshold or a changing frequency of the electric signal is larger than a preset frequency.

### CROSS-REFERENCE TO RELATED APPLICATION

This application claims a priority to Chinese Patent Application No. 201510369810.9 filed on Jun. 29, 2015, the disclosures of which are incorporated in their entirety by reference herein.

### TECHNICAL FIELD

The present disclosure relates to the field of daily-use articles, and in particular to a wrapping structure.

### BACKGROUND

A newborn has a weak immunocompetence and sensitive to the surrounding environment and temperature. In order to keep warm, the Chinese is used to put the newborn into a wrapping structure (i.e., a swaddle). In the actual situation, the swaddle is often made very thick to keep warm for the newborn. However the newborn will sweat due to the high temperature if the swaddle is too thick. Therefore, the newborn will be in a humid environment, and thus eczema may occur. As a result, people may make the swaddle thinner, and then the newborn may catch cold. Especially in the north part of China where a day-night temperature difference is large and the temperature at night is not uniform, the newborn may feel uncomfortable accordingly. In view of the above situation, it is very distressed for the parents to make the newborn to feel comfortable in the swaddle.

In the related art, a swaddle capable of controlling temperature is provided, so as to adjust the temperature within the swaddle according to the environment temperature. However, physiques of the newborns are different, and the newborns may have different feelings for the same temperature, thus it will not be determined whether the newborn feels comfortable using such swaddle. Therefore, the above issue may not be solved fundamentally.

### SUMMARY

An object of the present disclosure is to provide a wrapping structure, so as to determine whether the wrapped active object feels comfortable based on a body response of the active object.

A wrapping structure is provided, including a wrapping material configured to wrap an active object, where the wrapping material includes a detection layer configured to monitor a motion of the active object wrapped by the wrapping material and convert the motion of the active object into an electric signal, where an amplitude value of the electric signal is in direct proportion to a range and a frequency of the motion of the active object; the detection layer determines that the active object feels uncomfortable and sends a detection processing instruction, in the case that the amplitude value of the electric signal is not corresponding to a predetermined threshold or a changing frequency of the electric signal is larger than a preset frequency.

Optionally, the detection layer includes:

a signal transmission end, configured to transmit signals of a predetermined frequency;

a signal receiving end, configured to receive the signals transmitted by the signal transmission end;

a retractable metal grid layer, arranged between the signal transmission end and the signal receiving end and configured to shield a part of the signals transmitted from the signal transmission end to the signal receiving end, where a degree of signal shielding is based on a state of a deformation of the metal grid layer; and

a signal analysis circuit, connected to the signal receiving end and configured to acquire the electric signal from the signal receiving end and compare the acquired electric signal with the predetermined threshold, where the signal analysis circuit determines that the active object feels uncomfortable and sends the detection processing instruction in the case that the amplitude value of the electric signal is not corresponding to the predetermined threshold or the changing frequency of the electric signal is larger than the preset frequency.

The signal transmission end includes a signal generation source and a plurality of transmitting electrodes connected to the signal generation source; the signal receiving end includes a plurality of receiving electrodes which is in a one-to-one correspondence to the transmitting electrodes and parallel to the transmitting electrodes; the signal analysis circuit is connected to each of the receiving electrodes.

Optionally, the detection layer further includes a first base material made of a retractable insulating material and a second base material made of a retractable insulating material; the transmitting electrodes are arranged on a first surface of the first base material; the receiving electrodes are arranged on a second surface of the second base material; the first surface is parallel and opposite to the second surface.

Optionally, the retractable insulating material is arranged between the transmitting electrodes and the metal grid layer and between the receiving electrodes and the metal grid layer.

Optionally, the detection layer further includes a third base material made of a retractable insulating material, and the metal grid layer is arranged within the third base material.

Optionally, a signal gating circuit is arranged between each receiving electrode and the signal analysis circuit and configured to connect the receiving electrodes to the signal analysis circuit in turn by a predetermined frequency.

Optionally, the signal generation source outputs square-wave voltage signals or sine-cosine voltage signals.

Optionally, the wrapping material further includes a skin-friendly material layer which is cohesive to the detection layer.

Optionally, the wrapping material further includes a temperature adjustment layer which is cohesive to the detection layer, where the temperature adjustment layer functions in at least two thermal insulation states. The wrapping structure further includes a first controller which is configured to receive the detection processing instruction so as to make the temperature adjustment layer to adjust the thermal insulation state in response to the detection processing instruction.

Optionally, the first controller includes:

a first calculation chip, configured to determine a position where the electric signal is generated in the detection layer in response to the detection processing instruction;

an instruction transmission chip, configured to send information of the position to the temperature adjustment layer so as to adjust the thermal insulation state of a portion of the temperature adjustment layer corresponding to a region of the position where the electric signal is generated in response to the detection processing instruction.

Optionally, the wrapping structure further includes an alarm device configured to receive the detection processing instruction and generate an alarm signal.

Optionally, the temperature adjustment layer includes: a first flexible material with a plurality of air holes and a plurality of second flexible materials arranged on the first flexible material, where shapes and structures of the second flexible materials are corresponding to shapes and structures of the air holes and capable of translating relative to the first flexible material. A conductive line is arranged in each second flexible material to electrically connect the corresponding second flexible material to the first controller, and the second flexible material is driven to switch between a first state of covering the corresponding air hole and a second state of exposing the corresponding air hole in response to a control instruction of the first controller.

Optionally, the temperature adjustment layer includes: a third flexible material and a thermal-conductive resistor with a predetermined temperature in the third flexible material. The thermal-conductive resistor is electrically connected to the first controller, and a temperature of the thermal-conductive resistor is changed in response to the control instruction of the first controller.

According to the wrapping structure in some embodiments of the present disclosure, the detection layer monitors the motion of the wrapped active object and converts the motion of the active object into the electric signal. The range and the frequency of the motion of the active object are reflected by the amplitude value of the electric signal. Therefore, it is able to determine whether the wrapped active object feels comfortable based on a body response of the active object. In the case that the active object is a newborn, a person taking care of the newborn may be able to determine whether the newborn feels comfortable using such wrapping structure.

### BRIEF DESCRIPTION OF THE DRAWINGS

FIG. 1 is a brief schematic view showing a wrapping structure in some embodiments of the present disclosure;

FIG. 2 is a sectional view of a detection layer of a wrapping structure in some embodiments of the present disclosure;

FIG. 3 is a plan view of a detection layer of a wrapping structure in some embodiments of the present disclosure:

FIG. 4 is a schematic view showing a portion of a temperature adjustment layer of a wrapping structure in some embodiments of the present disclosure; and

FIG. 5 is a schematic view showing a wrapping structure in some embodiments of the present disclosure.

### DETAILED DESCRIPTION

In order to make the objects, the technical solutions and the advantages of the present disclosure more apparent, the present disclosure will be described hereinafter in conjunction with the drawings and embodiments.

A wrapping structure is provided in some embodiments of the present disclosure, including a wrapping material configured to wrap an active object. The wrapping material includes a detection layer configured to monitor a motion of the active object wrapped by the wrapping material and convert the motion of the active object into an electric signal. An amplitude value of the electric signal is in direct proportion to a range and a frequency of the motion of the active object. The detection layer determines that the active object feels uncomfortable and sends a detection processing instruction, in the case that the amplitude value of the electric signal is not corresponding to a predetermined threshold or a changing frequency of the electric signal is larger than a preset frequency.

In the above wrapping structure, the detection layer monitors the motion of the wrapped active object and converts the motion of the active object into the electric signal. The range and the frequency of the motion of the active object are reflected by the amplitude value of the electric signal. When the amplitude value of the electric signal is not corresponding to the predetermined threshold or the changing frequency of the electric signal is larger than the preset frequency, it is indicated that the active object motions acutely due to the uncomfortable feeling. In such case, the detection processing instruction is sent out, so as to alert the parents to handle this issue or adjust the temperature within the wrapping structure by themselves. Therefore, according to the wrapping structure provided in the present disclosure, it is able to determine whether the wrapped active object feels comfortable based on a body response of the active object, so as to facilitate a person taking care of the active object to determine whether the active object feels comfortable. For example, when the active object is a newborn, the parents of the newborn will determine whether the newborn feels comfortable by using such wrapping structure.

In some embodiments of the present disclosure, the wrapping structure may be configured to wrap a newborn as a swaddle. Of course, the wrapping structure may also be configured to wrap other active object besides the newborn, such as an old people who is sick in bed and cannot take care of himself.

FIG. 1 is a brief schematic view showing a wrapping structure in some embodiments of the present disclosure, which is used as a swaddle. The wrapping structure provided in some embodiments of the present disclosure includes at least two layers.

Referring to FIG. 1, a wrapping material of the wrapping structure includes a detection layer 102 and a skin-friendly material layer 101 which is cohesive to the detection layer 102. The skin-friendly material layer 101 is a made of a soft cloth. When a newborn is wrapped by the wrapping structure, the skin-friendly material layer 101 may be arranged at an inner side of the wrapping structure and contact the newborn directly, so as to make the newborn to feel comfortable in the wrapping structure as a swaddle. Optionally, the skin-friendly material layer 101 is detachably jointed to the detection layer 102 via, for example, velcro backings. By such detachable junction therebetween, the skin-friendly material layer 101 may be replaced timely based on the environment temperature. When the environment temperature is relatively low, it is able to make the skin-friendly material layer 101 by a relative thick fabric. When the environment temperature is relatively high, it is able to make the skin-friendly material layer 101 by a relative thin fabric. Therefore, it is able to meet the requirements of using the wrapping structure in different temperatures as a swaddle for the newborn.

Optionally, referring to FIG. 1, the wrapping material of the wrapping structure in some embodiments of the present disclosure further includes a temperature adjustment layer 103 which is cohesive to the detection layer 102. In some embodiments of the present disclosure, when an active object is wrapped, the temperature adjustment layer 103 is arranged at a side of the detection layer 102 away from the active object, so as to adjust a thermal insulation state of the wrapping material. Of course, it is not limited to arrange the temperature adjustment layer 103 at a side of the detection layer 102 away from the active object. For example, the temperature adjustment layer 103 may also be arranged at an inner side of the detection layer 102 and between the detection layer 102 and the skin-friendly material layer 101.

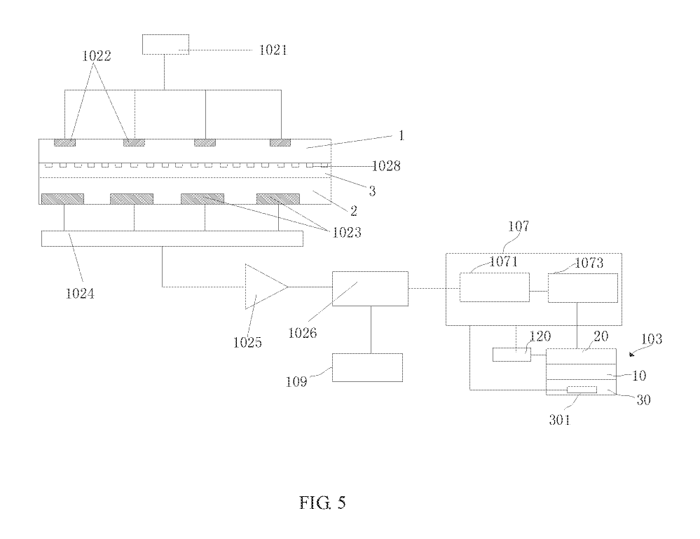

In some embodiments of the present disclosure, the temperature adjustment layer 103 functions in at least two thermal insulation states. The wrapping structure further includes a first controller 107 (as shown in FIG. 5). The first controller is connected to both the detection layer 102 and the temperature adjustment layer 103, so as to receive a detection processing instruction sent by the detection layer 102 in the case that the detection layer 102 determines that the active object feels uncomfortable. The first controller 107 sends an adjustment instruction to the temperature adjustment layer 103 in response to the detection processing instruction, and makes the temperature adjustment layer 103 to adjust the thermal insulation state, so as to guarantee the wrapping structure to be capable of automatically adjusting the temperature within the wrapping structure to a temperature suitable for the active object in response to the uncomfortable feeling of the active object.

The structures of the detection layer 102 and the temperature adjustment layer 103 will be described in details in conjunction with embodiments.

In some embodiments of the present disclosures, the detection layer 102 includes:

a signal transmission end, configured to transmit a signal of a predetermined frequency:

a signal receiving end, configured to receive the signal transmitted by the signal transmission end;

a retractable metal grid layer, arranged between the signal transmission end and the signal receiving end and configured to shield a part of the signals transmitted from the signal transmission end to the signal receiving end, where a degree of signal shielding is based on a state of a deformation of the metal grid layer;

a signal analysis circuit, connected to the signal receiving end and configured to acquire an electric signal of the signal receiving end and compare the acquired electric signal with the predetermined threshold, where the signal analysis circuit determines that the active object feels uncomfortable and sends the detection processing instruction in the case that the amplitude value of the electric signal is larger than the predetermined threshold.

The retractable metal grid layer is arranged between the signal transmission end and the signal receiving end of the above detection layer 102. When the signal transmission end transmits signals of a predetermined frequency to the signal receiving end, the retractable metal grid layer therebetween is able to shield a part of the signals transmitted therebetween. In such case, when the retractable metal grid layer is not subjected to a deformation, the grids of the grid layer are of a predetermined size, the amplitude value of the shielded signal is stable; or when the retractable metal grid layer is subjected to a deformation, the grids of the grid layer are changed, i.e., the grids are contracted or enlarged, the amplitude value of the shielded signal is changed accordingly. Based on the above, the signal analysis circuit is connected to the signal receiving end, so as to acquire the electric signals of the signal receiving end and then compare the acquired electric signals with a signal of a stable amplitude value (i.e., the predetermined threshold). When the amplitude value of the acquired electric signal is larger than the predetermined threshold by a preset value or smaller than the predetermined threshold by a preset value, it is determined that the received electric signal is not corresponding to the predetermined threshold. In addition, the signal analysis circuit is further configured to monitor a changing frequency of the amplitude value of the acquired signal relative to the predetermined threshold so as to determine whether a changing frequency of the electric signal is larger than a preset frequency.

The above detection layer 102 is applied in the wrapping structure, where the retractable metal grid layer is tiled in the wrapping material as a whole. The retractable metal grid layer wraps every portion of the active object. Optionally, the grids of the retractable metal grid layer are of an identical size. When the active object is in a resting state in the wrapping structure, the motion of the active object is tiny, then the amplitude value of the electric signal acquired by the signal analysis circuit is corresponding to the predetermined threshold, and thus it is determined that the active object feels comfortable. When the human body motions in the wrapping structure, the motion range of the active object is big, then the amplitude value of the acquired electric signal is larger than the predetermined threshold by a preset value or smaller than the predetermined threshold by a preset value, and thus it is determined that the received electric signal is not corresponding to the predetermined threshold and the active object feels uncomfortable; or when the received electric signal changes very frequently and a changing frequency thereof is larger than the preset frequency, it is also determined that the active object feels uncomfortable, and then the signal analysis circuit sends out a detection processing instruction.

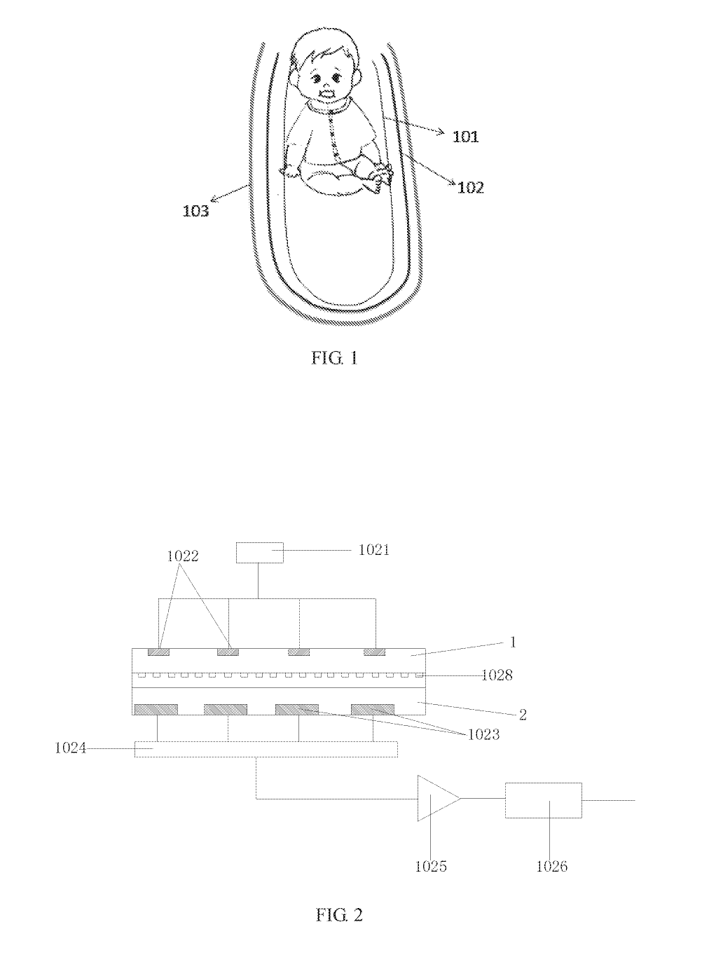

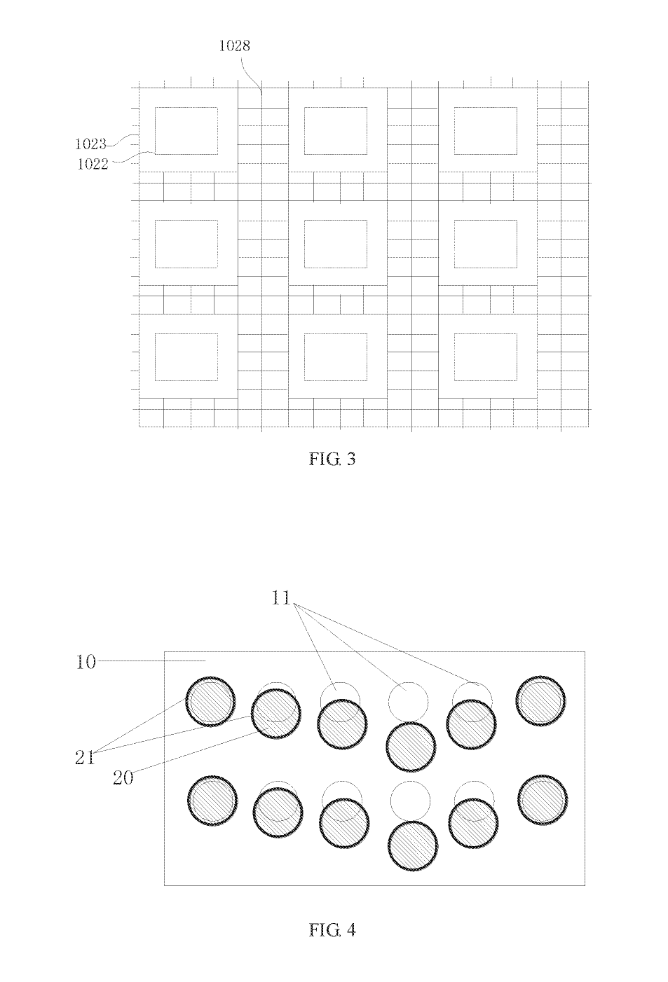

FIG. 2 is a sectional view of a detection layer of a wrapping structure in some embodiments of the present disclosure. FIG. 3 is a plan view of the detection layer of the wrapping structure in some embodiments of the present disclosure.

Referring to FIGS. 2 and 3, the signal transmission end of the detection layer 102 includes a signal generation source 1021 and a plurality of transmitting electrodes 1022 connected to the signal generation source 1021. The signal receiving end includes a plurality of receiving electrodes 1023 which are in a one-to-one correspondence to the transmitting electrodes 1022 and parallel to the transmitting electrodes 1022. A tiled retractable metal grid layer 1028 is arranged between the transmitting electrodes 1022 and the receiving electrodes 1023. Optionally, the metal grids of the retractable metal grid layer 1028 are of an identical size, and the signal analysis circuit is connected to each of the receiving electrodes 1023.

Optionally, the signal generation source 1021 outputs by a first predetermined frequency square-wave voltage signals or sine-cosine voltage signals.

The above detection layer 102 includes a plurality of transmitting electrodes 1022 and a plurality of receiving electrodes 1023. Because the transmitting electrodes 1022 are electrically connected to the signal generation source 1021, an electric field is formed between the transmitting electrodes 1022 and the receiving electrodes 1023 and a coupling capacitor is formed there between. The retractable metal grid layer 1028 is arranged in the formed electric field. Optionally, the retractable metal grid layer 1028 is grounded. The retractable metal grid layer 1028 shields the electric field between the transmitting electrodes 1022 and the receiving electrodes 1023 by a certain degree and functions as a capacitor. The sparser the grids of the retractable metal grid layer 1028 are, the larger the capacitance thereof will be. The denser the grids of the retractable metal grid layer 1028 are, the smaller the capacitance thereof will be. Furthermore, the larger the capacitance of the retractable metal grid layer 1028 is, the larger a quantity of electric change of the receiving electrodes 1023 will be, and the larger the amplitude value of the electric signal acquired by the signal analysis circuit from the receiving electrodes 1023 will be. On the contrary, the smaller the capacitance of the retractable metal grid layer 1028 is, the smaller a quantity of electric change of the receiving electrodes 1023 will be, and the smaller the amplitude value of the electric signal acquired by the signal analysis circuit from the receiving electrodes 1023 will be.

The above description is merely an embodiment of the detection layer 102 configured to convert the motion of the active object into the electric signal, but the present disclosure is not limited herein. Those skilled in the art may realize the function of the above detection layer 102 by using the other structures in the related art based on the principle of the present disclosure.

In some embodiment of the present disclosure, a signal gating circuit 1024 is arranged between each receiving electrode 1023 and the signal analysis circuit and configured to connect the receiving electrodes 1023 to the signal analysis circuit in turn by a second predetermined frequency. The electric signal of the receiving electrode 1023 selected to be connected to the signal analysis circuit is transmitted to the signal analysis circuit.

Referring to FIG. 2, the signal analysis circuit includes an amplifier 1025 configured to acquire the electric signal of the receiving electrode 1023 selected to be connected to the signal analysis circuit. The signal analysis circuit further includes a processing chip 1026 connected to the amplifier 1025 which is configured to compare the amplitude value of the electric signal output by the amplifier 1025 with the predetermined threshold. When the amplitude value of the electric signal is larger than the predetermined threshold by a preset value or smaller than the predetermined threshold by a preset value, it will be determined that the received electric signal is not corresponding to the predetermined threshold and the active object feels uncomfortable; or when the received electric signal changes very frequently and a changing frequency thereof is larger than the preset frequency, it is also determined that the active object feels uncomfortable, and then the processing chip 1026 sends out a corresponding detection processing instruction.

In addition, referring to FIG. 3, in some embodiments of the present disclosure, the transmitting electrodes 1022 and the receiving electrodes 1023 are both of a block-like structure. Optionally, an area of each receiving electrode 1023 is larger than that of each transmitting electrode 1022. Furthermore, the transmitting electrodes 1022 and the receiving electrodes 1023 are arranged at two sides of the retractable metal grid layer 1028 respectively.

Furthermore, referring to FIG. 2, the transmitting electrodes 1022 are distributed uniformly on a first surface of a first base material 1 made of a retractable insulating material, and the receiving electrodes 1023 are distributed uniformly on a second surface of a second base material 2 made of a retractable insulating material. The first surface is parallel and opposite to the second surface. Optionally, the retractable insulating material is arranged between the transmitting electrodes 1022 and the retractable metal grid layer 1028 and between the receiving electrodes 1023 and retractable the metal grid layer 1028, so as to make the transmitting electrodes 1022, the retractable metal grid layer 1028 and the receiving electrodes 1023 to be insulated from one another. Optionally, in order to make the transmitting electrodes 1022, the retractable metal grid layer 1028 and the receiving electrodes 1023 to be insulated from one another, the retractable metal grid layer 1028 may be arranged within a third base material 3 (shown in FIG. 5) made of the retractable insulating material.

Optionally, according to the wrapping structure in some embodiments of the present disclosure, the receiving electrodes 1023 are selected to be connected to the signal analysis circuit in turn via the corresponding signal gating circuits 1024, the electric signals of the receiving electrodes 1023 are transmitted to the signal analysis circuit in turn, and then the signal analysis circuit may determine the receiving electrode 1023 from which each electric signal is transmitted. When the signal analysis circuit sends out the detection processing instruction, it may send to the first controller the information of the receiving electrode 1023 whose electric signal is of an amplitude value which is not corresponding to the predetermined threshold or has a relative large changing frequency, so as to make the first controller to adjust the thermal insulation state of a region of the temperature adjustment layer 103 corresponding to the determined receiving electrode 1023.

Therefore, the wrapping structure in some embodiments of the present disclosure further includes a first controller 107 which is connected to the processing chip 1026 shown in FIG. 2. As shown in FIG. 5, the first controller 107 includes:

a first calculation chip 1071, configured to determine a position where the electric signal is generated in the detection layer in response to the detection processing instruction sent by the processing chip 1026:

an instruction transmission chip 1073, configured to send information of the position to the temperature adjustment layer 103 so as to adjust the thermal insulation state of a portion of the temperature adjustment layer 103 corresponding to a region of the position where the electric signal is generated in response to the detection processing instruction.

Optionally, the wrapping structure further includes an alarm device 109 configured to receive the detection processing instruction sent by the processing chip 1026 and generate an alarm signal, so as to alert the parents of the wrapped active object to handle the uncomfortable feeling of the active object. According to the wrapping structure in some embodiments of the present disclosure, the temperature adjustment may be controlled by the temperature adjustment layer 103. In order to reduce the user's workload, the temperature adjustment layer may adjust the temperature automatically in response to the detection processing instruction after processing chip 1026 sends the detection processing instruction for the first time after a predetermined time period. Only when the processing chip 1026 sends out the detection processing instruction for a plurality of times and the temperature adjustment layer 103 adjusts the temperature accordingly for a plurality of times (e.g., one to three times), and then the amplitude value of the electric signal is not corresponding to the predetermined threshold or the changing frequency of the electric signal is larger than the preset frequency, the alarm will be sent again to alert the parents of the wrapped active object.

The temperature adjustment layer 103 in some embodiments of the present disclosure may include a first flexible material with a plurality of air holes and a plurality of second flexible materials arranged on the first flexible material. Shapes and structures of the second flexible materials are corresponding to shapes and structures of the air holes and capable of translating relative to the first flexible material. A conductive line is arranged in each second flexible material to electrically connect the corresponding second flexible material to the first controller. The second flexible material is driven to switch between a first state of covering the corresponding air hole and a second state of exposing the corresponding air hole in response to a control instruction of the first controller.

FIG. 4 is a schematic view showing a portion of a temperature adjustment layer of a wrapping structure in some embodiments of the present disclosure. The first flexible material 10 has a plurality of air holes 11, and the shapes and structures of the second flexible materials 20 are corresponding to the shapes and structures of the air holes, and a support 21 with a certain strength is arrange at a periphery of each of the second flexible materials 20. Each support 21 may be connected to a drive structure 120 which is electrically connected to the first controller by the conductive line. The drive structure drives the corresponding support 21 to move in response to a control instruction sent by the first controller, so as to translate the corresponding second flexible material 20, and then the area of the air hole 11 covered by the second flexible material 20 may be increased or reduced, thereby changing the thermal insulation state of the temperature adjustment layer 103. When the area of the air hole 11 covered by the second flexible material 20 is small, a breathability is good while the thermal insulation is not good. When the area of the air hole 11 covered by the second flexible material 20 is big, a breathability is not good while the thermal insulation is good. In some embodiments of the present disclosure, the drive structure 120 may be any drive structure such as electrically-driven structure or mechanically-driven structure.

The temperature adjustment layer 103 in some embodiments of the present disclosure may further include a third flexible material 30 and a thermal-conductive resistor 301 with a predetermined temperature in the third flexible material 30. The thermal-conductive resistor is electrically connected to the first controller, and a temperature of the thermal-conductive resistor is changed in response to the control instruction of the first controller.

The above temperature adjustment layer 103 is of a structure like an electric blanket. A thermal-conductive resistor is arranged in the third flexible material, and the thermal insulation state of the temperature adjustment layer 103 is adjusted based on a temperature change of the thermal-conductive resistor.

The above two kinds of structures of the temperature adjustment layer 103 are merely the preferred embodiments, and the present disclosure is not limited herein. A person skilled in the art may make improvements to these embodiments in accordance with the basic concept of the present disclosure.

According to the wrapping structure in some embodiments of the present disclosure, the detection layer monitors the motion of the wrapped active object and converts the motion of the active object into the electric signal. The range and the frequency of the motion of the active object are reflected by the amplitude value of the electric signal. Therefore, it is able to determine whether the wrapped active object feels comfortable based on a body response of the active object, so as to facilitate a person taking care of the active object to determine whether the active object feels comfortable.

The above are merely the preferred embodiments of the present disclosure. A person skilled in the art may make further modifications and improvements without departing from the principle of the present disclosure, and these modifications and improvements shall also fall within the scope of the present disclosure.

1. A wrapping structure comprising:
a wrapping material configured to wrap an active object:
wherein the wrapping material comprises a detection layer configured to monitor a motion of the active object wrapped by the wrapping material and convert the motion of the active object into an electric signal, and an amplitude value of the electric signal is in direct proportion to a range and a frequency of the motion of the active object;
wherein the detection layer determines that the active object feels uncomfortable and sends a detection processing instruction in the case that the amplitude value of the electric signal is not corresponding to a predetermined threshold or a changing frequency of the electric signal is larger than a preset frequency.

2. The wrapping structure according to claim 1, wherein the detection layer comprises:
a signal transmission end, configured to transmit signals of a predetermined frequency;
a signal receiving end, configured to receive the signals transmitted by the signal transmission end;
a retractable metal grid layer between the signal transmission end and the signal receiving end, and configured to shield a part of the signals transmitted from the signal transmission end to the signal receiving end; wherein a degree of signal shielding is based on a state of a deformation of the metal grid layer; and
a signal analysis circuit, connected to the signal receiving end and configured to acquire the electric signal from the signal receiving end and compare the acquired electric signal with the predetermined threshold;
wherein the signal analysis circuit determines that the active object feels uncomfortable and sends the detection processing instruction in the case that the amplitude value of the electric signal is not corresponding to the predetermined threshold or the changing frequency of the electric signal is larger than the preset frequency.

3. The wrapping structure according to claim 2, wherein the signal transmission end comprises a signal generation source and a plurality of transmitting electrodes connected to the signal generation source;
the signal receiving end comprises a plurality of receiving electrodes which are in a one-to-one correspondence to the transmitting electrodes and parallel to the transmitting electrodes; and
the signal analysis circuit is connected to each of the receiving electrodes.

4. The wrapping structure according to claim 3, wherein the detection layer further comprises a first base material made of a retractable insulating material and a second base material made of a retractable insulating material;
the transmitting electrodes are on a first surface of the first base material;
the receiving electrodes are on a second surface of the second base material; and
the first surface is parallel and opposite to the second surface.

5. The wrapping structure according to claim 4, further comprising retractable insulating material between the transmitting electrodes and the metal grid layer and between the receiving electrodes and the metal grid layer.

6. The wrapping structure according to claim 2, wherein the detection layer further comprises a third base material made of a retractable insulating material, and the metal grid layer is arranged within the third base material.

7. The wrapping structure according to claim 3, further comprising a signal gating circuit between each of the receiving electrodes and the signal analysis circuit; the signal gating circuit is configured to connect the receiving electrodes to the signal analysis circuit in turn by a predetermined frequency.

8. The wrapping structure according to claim 3, wherein the signal generation source outputs square-wave voltage signals or sine-cosine voltage signals.

9. The wrapping structure according to claim 1, wherein the wrapping material further comprises a skin-friendly material layer which is cohesive to the detection layer.

10. The wrapping structure according to claim 1, wherein the wrapping material further comprises a temperature adjustment layer which is cohesive to the detection layer;
wherein the temperature adjustment layer functions in at least two thermal insulation states; and
wherein the wrapping structure further comprises a first controller which is configured to receive the detection processing instruction so as to make the temperature adjustment layer to adjust the thermal insulation state in response to the detection processing instruction.

11. The wrapping structure according to claim 10, wherein the first controller comprises:
a first calculation chip, configured to determine a position where the electric signal is generated in the detection layer in response to the detection processing instruction; and
an instruction transmission chip, configured to send information of the position to the temperature adjustment layer so as to adjust the thermal insulation state of a portion of the temperature adjustment layer corresponding to a region of the position where the electric signal is generated in response to the detection processing instruction.

12. The wrapping structure according to claim 1, further comprising an alarm device configured to receive the detection processing instruction and generate an alarm signal.

13. The wrapping structure according to claim 10, wherein the temperature adjustment layer comprises:
a first flexible material with a plurality of air holes and a plurality of second flexible materials on the first flexible material;
wherein shapes and structures of the second flexible materials are corresponding to shapes and structures of the air holes and capable of translating relative to the first flexible material; and
a conductive line is arranged in each of the second flexible materials to electrically connect the corresponding second flexible material to the first controller, and the second flexible material is driven to switch between a first state of covering the corresponding air hole and a second state of exposing the corresponding air hole in response to a control instruction of the first controller.

14. The wrapping structure according to claim 10, wherein the temperature adjustment layer comprises:
a third flexible material and a thermal-conductive resistor with a predetermined temperature in the third flexible material; wherein the thermal-conductive resistor is electrically connected to the first controller, and a temperature of the thermal-conductive resistor is changed in response to the control instruction of the first controller.

15. The wrapping structure according to claim 5, wherein the detection layer further comprises a third base material made of a retractable insulating material, and the metal grid layer is arranged within the third base material.
